(19) United States
(12) Patent Application Publication
(10) Pub. No.: US 2013/0304209 A1
(43) Pub. Date: Nov. 14, 2013

(54) BIOLOGIC PARTIAL MENISCUS AND METHOD OF PREPARATION

(71) Applicant: Arthrex, Inc., Naples, FL (US)

(72) Inventors: Reinhold Schmieding, Naples, FL (US); Brandon L. Roller, Naples, FL (US)

(21) Appl. No.: 13/866,079

(22) Filed: Apr. 19, 2013

Related U.S. Application Data
(60) Provisional application No. 61/645,799, filed on May 11, 2012.

(51) Int. Cl. A61F 2/30

(52) U.S. Cl. CPC A61F 2/30756
USPC 623/14.12; 29/527.1

(57) ABSTRACT

Biologic partial menisci (biologic partial meniscal replacements/constructs) used to replace at least a part of a meniscus, and methods of forming such biologic partial menisci. Meniscal cartilage is employed to form a moldable allograft paste. A sterile mold that replicates a meniscus (for example, the medial or lateral meniscus) is provided in various sizes and is used as a biologic mold to recreate the anatomic shape of the meniscus. The moldable paste is inserted (for example, injected) into the mold and allowed to set into a stable, anatomically shaped meniscus (the biologic partial meniscus). Fibrin glue or other biologic adhesives or strengtheners may be optionally added to provide further biomechanical strength. Once the biologic partial meniscus is removed from the mold, it is provided at the surgical site and attached to the excised meniscus (placed into the correct anatomical shape) to complete the meniscal repair.

### CROSS-REFERENCE TO RELATED APPLICATIONS

This application claims the benefit of U.S. Provisional Application No. 61/645,799, filed May 11, 2012, the disclosure of which is incorporated by reference in its entirety herein.

### FIELD OF THE INVENTION

The present invention relates to the field of surgery and, more particularly, to an improved partial meniscus implant and improved partial meniscectomy repairs.

### BACKGROUND OF THE INVENTION

Loss or tear of meniscal tissue leads to increased pain and decreased clinical function and activity levels. Meniscectomy is the surgical removal of all or part of a torn meniscus. In partial meniscectomy, only a part of the meniscus is removed (i.e., only the unstable meniscal fragments) and the remaining meniscus edges are smoothed so that there are no frayed ends.

No viable substitute for a partially resected meniscus is currently available in the United States. Meniscal allografts are known and used primarily as a complete meniscus replacement, and a polyurethane meniscus (a polyurethane scaffold) has been used for total and partial replacement of the meniscus but with limited results.

A need exists for a biologic allograft based alternative to rebuild a resected partial meniscus through an arthroscopic procedure. Also needed is a method of surgery employing a partial meniscus consisting essentially of a biological material, and a method of preparing a biologic partial meniscal replacement.

### BRIEF SUMMARY OF THE INVENTION

The present invention provides partial meniscus components, implements and techniques for arthroscopic surgical repairs such as partial meniscal repairs.

The present invention also provides a biologic partial meniscus (biologic partial meniscal replacement/construct) used to replace at least a part of a meniscus, and methods of forming such biologic partial meniscus. Meniscal cartilage is employed to form a moldable allograft paste. A sterile mold that replicates at least a portion or a full meniscus (for example, the medial or lateral meniscus) is provided in various sizes and is used as a biologic mold to recreate the anatomic shape of the meniscus. The moldable paste is provided into the mold and allowed to set into a stable, anatomically shaped meniscus (the biologic partial meniscus). Optionally, flexible strands such as sutures or a collagen mesh may be added into the mold prior to the insertion of the paste into the mold, to create a biologic reinforcement to strengthen the construct (the biologic partial meniscus). Fibrin glue or other biologic adhesives or strengtheners may be optionally added to provide further biomechanical strength. Once the biologic partial meniscus is removed from the mold, it is provided at the surgical site and attached to the excised meniscus (placed into the correct anatomical shape) to complete the meniscal repair. The biologic partial meniscus may be provided at the surgical site by various arthorscopically-assisted surgical techniques for partial meniscal repairs.

Other features and advantages of the present invention will become apparent from the following description of the drawings and the invention.

### BRIEF DESCRIPTION OF THE DRAWINGS

FIG. 1 illustrates a schematic illustration of a front view of a knee with a meniscus with a partial tear.

FIG. 2 illustrates a top view of the meniscus of FIG. 1.

FIG. 3 illustrates an exemplary embodiment of a mold for partial meniscal repair according to the present invention.

FIG. 4 illustrates the mold of FIG. 3 with a moldable paste provided within at least a region of the mold.

FIG. 5 illustrates a biologic partial meniscus (BioMeniscus or graft) of the present invention (removed from the mold of FIG. 4).

FIG. 6 illustrates a top view of the meniscus of FIG. 1 with the torn portion/region excised.

FIG. 7 illustrates a top view of the meniscus of FIG. 6 repaired by a method of the present invention (with the biologic partial meniscus (BioMeniscus or graft) of FIG. 5 provided at the repair site).

### DETAILED DESCRIPTION OF THE INVENTION

The present invention provides partial meniscus components, implements and techniques for arthroscopic surgical repairs such as partial meniscal repairs.

The present invention also provides biologic partial menisci (biologic partial meniscal replacements/constructs) used to replace at least a part of a meniscus, and methods of forming such biologic partial menisci.

As detailed below, meniscal cartilage is employed to form a moldable allograft paste or composition. Meniscal cartilage (in the form of morsellized, freeze-dried and/or desiccated meniscal cartilage, for example) may be processed by a tissue bank similar to the BioCartilage™ process for hyaline cartilage. This sterile, freeze-dried and/or desiccated product may be mixed (by the orthopedic surgeon, for example) at the time of surgery with ACP, CACL or an autograft biologic equivalent to create a moldable allograft paste.

A sterile mold that replicates the medial or lateral meniscus in various sizes is used as a biologic mold to recreate the anatomic shape of the meniscus. The moldable allograft paste/composition is provided into the mold (for example, by being inserted or injected into the mold, or by any other known methods) and allowed to set until the ACP coagulates into a stable, anatomically shaped meniscus (biologic partial meniscus or biologic partial meniscal replacement/construct). Sutures or a collagen mesh may be added into the mold prior to the paste being injected, to create a biologic reinforcement to strengthen the construct. Fibrin glue or other biologic adhesives or strengtheners may be added to provide further biomechanical strength. Optionally, flexible strands such as sutures may extend from the set biological construct to aid in the fixation of the construct at the repair site.

The biologic partial meniscus is removed from the mold. The section for the meniscus required for anatomic partial replacement is excised and pre-stitched for graft passing and fixation. A cannulated, vented stent may be inserted in the remaining rim of the meniscus and the biologic partial meniscus inserted over the stents as fixation posts that provide additional vascularity to the biologic partial meniscus graft from the red zone of the remaining meniscal rim.

Additional suturing and/or stitching (such as, for example, meniscal cinch and/or alternative all-inside sutures) may be used to provide further fixation and mechanical stability of the biologic partial meniscus at the surgical site. Placing the knee in full extension and a post-op brace that maintains full extension creates an anatomic force to the biologic partial meniscus graft to further shape it into the correct anatomic shape during final coagulation and solidification.

The present invention also provides biologic partial menisci or biologic partial meniscal replacements/constructs and methods of preparing such replacements/constructs, as well as methods of meniscal reconstruction with a biologic partial meniscus or biologic partial meniscal replacement/construct. The invention also provides arthroscopically-assisted surgical techniques for partial meniscal repairs.

Although the use of the biologic partial meniscus of the present invention will be described below with reference to a particular application, i.e., an arthroscopic knee application, it must be understood that the application is not limited to this exemplary-only embodiment. Accordingly, the invention also contemplates additional applications, for example, meniscal hip or shoulder applications, among many other meniscal repair applications.

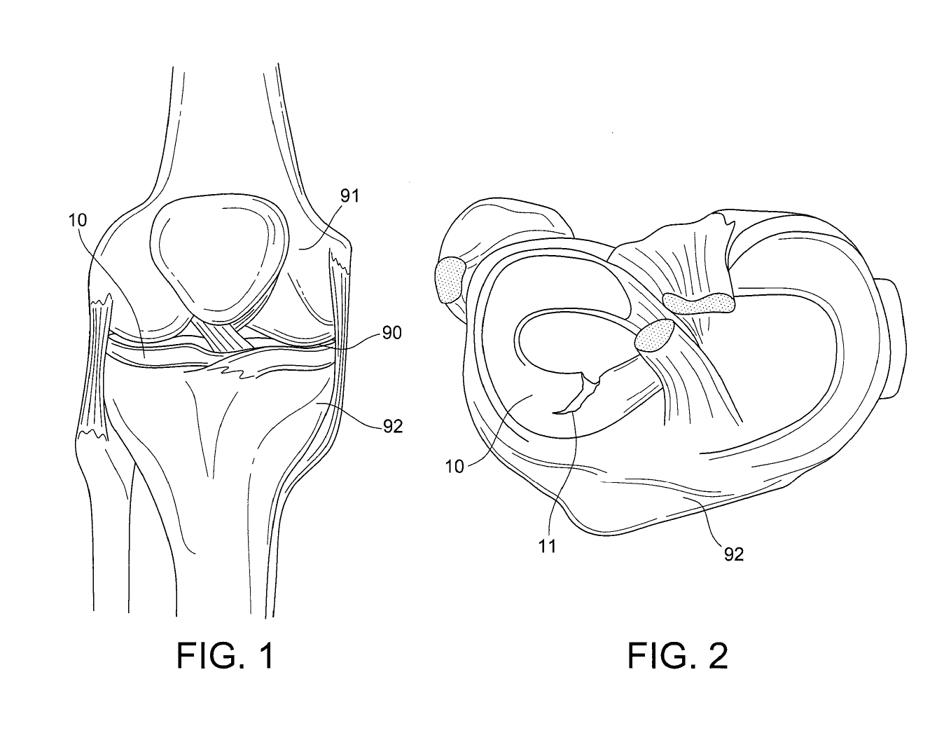

Referring now to the drawings, where like elements are designated by like reference numerals, FIGS. 1 and 2 illustrate schematic representations of a knee joint 90 with an exemplary torn meniscus 10 provided between femur 91 and tibia 92. FIG. 2 illustrates a top view of torn meniscus 10 of FIG. 1, with exemplary tear/lesion 11 which is excised and replaced with a biologic partial meniscus 50 or biologic partial meniscal replacement/construct 50 of the present invention, and as detailed below.

In an exemplary embodiment, the biologic partial meniscus 50 may be formed of meniscal cartilage (preferably morcellized or desiccated) which may be processed by a tissue bank similar to the BioCartilage™ process for hyaline cartilage.

According to an exemplary-only embodiment, BioCartilage™ or a similar material is employed to provide a reproducible, simple and inexpensive method to augment tissue repair procedures. BioCartilage™ consists essentially of allograft cartilage that has been dehydrated and micronized. BioCartilage™ contains the extracellular matrix that is native to articular cartilage including key components such as type II collagen, proteoglycans, and additional cartilaginous growth factors. The principle of BioCartilage™ is to serve as a scaffold over a defect providing a tissue network that can potentially signal autologous cellular interactions and improve the degree and quality of tissue healing within a properly prepared meniscal defect.

The sterile, freeze-dried or desiccated cartilage is mixed (by the orthopedic surgeon, for example) at the time of surgery with ACP, CACL, PRP, fibrin glue or an autograft biologic equivalent, to create a moldable allograft paste 60.

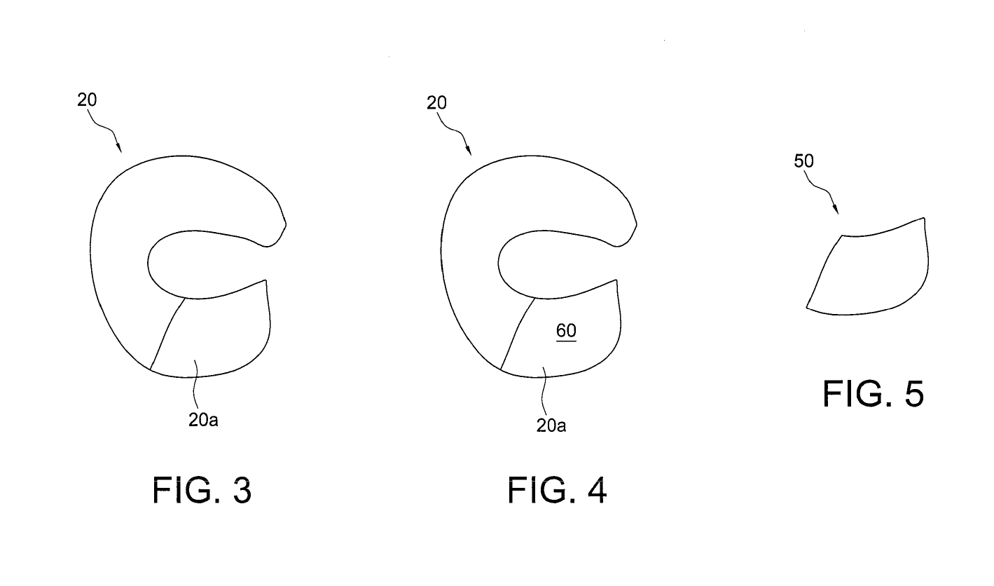

Once the torn region 11 of the partial meniscus 10 is removed (i.e., once the unstable meniscal fragments 11 are removed) and the remaining meniscal edges are cleaned and smooth, a sterile mold 20 is employed to replicate the medial or lateral meniscus to be repaired. For example, FIG. 3 illustrates an exemplary mold 20 that mimics the configuration and dimensions of partial torn meniscus 10 of FIGS. 1 and 2. Mold 20 may be provided in various sizes and is used as a biologic mold to recreate the anatomic shape of the meniscus to be repaired. Mold 20 may comprise a clear or plastic material, or a biocompatible metal, or a combination of plastic and additional materials.

The paste 60 is provided (for example, inserted or injected) into the mold and allowed to set until the ACP coagulates into a stable, anatomically-shaped meniscus 50 (FIG. 5). FIG. 4 illustrates paste 60 provided within mold area/region 20a of the mold 20. Mold region 20a resembles the resected meniscal area 11 of the torn meniscus 10 of FIGS. 1 and 2. In an exemplary embodiment, mold 20 is a reconstruction/resurfacing mold that reproduces only a part of the meniscus, for example, only the torn/damaged area that needs to be replaced.

At least one flexible strand (for example, one or more sutures or combinations of sutures and additional strands) or a collagen mesh may be added (if desired) into the mold 20 and preferably prior to the paste 60 being injected, to create a biologic reinforcement to strengthen the construct 50. Fibrin glue or other biologic adhesives or strengtheners may be also added to provide further biomechanical strength.

Additionally, at least one flexible strand may be provided into the mold preferably before the injection of the paste into the mold, so that at least a portion of the flexible strand extends from the set biological construct. The at least one flexible strand may include suture such as FiberWire® suture (disclosed in U.S. Pat. No. 6,716,234) or TigerWire® suture, or suture tape (such as FiberTape® disclosed in U.S. Pat. No. 7,892,256), among others, or combination of these materials. The at least one flexible strand may include strands of various colors to aid in the surgical repair.

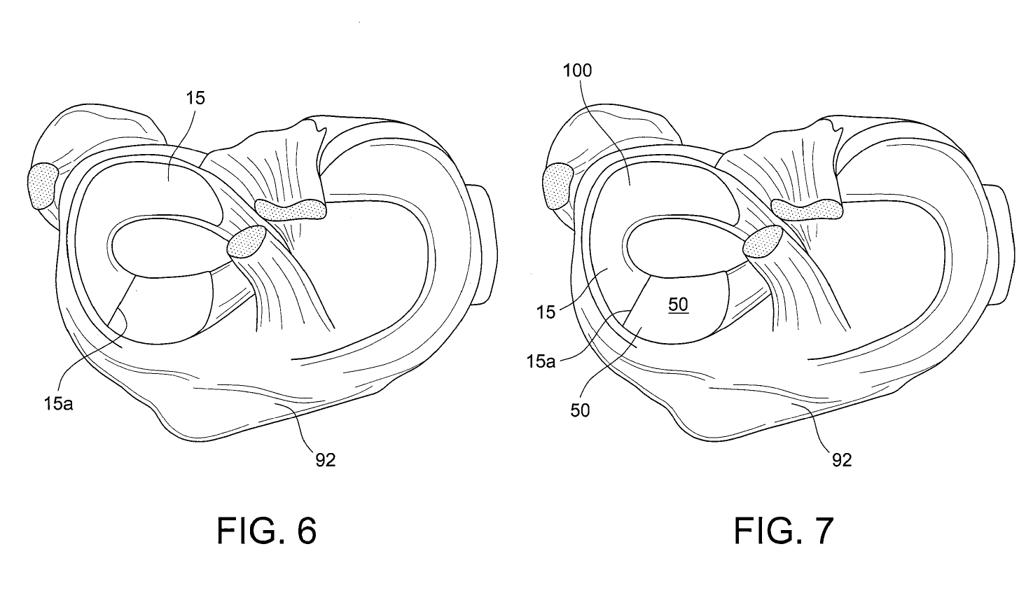

The biologic meniscal construct 50 (FIG. 5) is removed from the mold 20. The section 11 of the meniscus 10 required for anatomic partial replacement is excised and the remaining edge 15a of the remaining meniscus 15 (excised meniscus 15 of FIG. 6) is pre-stitched for graft passing and fixation. A cannulated, vented stent may be inserted in the remaining rim 15a of the meniscus 15 and the construct 50 is inserted over the stents as fixation posts that provide additional vascularity to the graft 50 from the red zone of the remaining meniscal rim. Various fixation methods and techniques such as suturing, stitching, meniscal cinch and/or alternative all-inside sutures may be used to provide further fixation and mechanical stability of the biologic partial meniscus 50 at the surgical site, to complete the meniscal repair or repaired/reconstructed meniscus 100 (FIG. 7).

Placing the knee in full extension and a post-op brace that maintains full extension may be employed to create an anatomical force to the biologic partial meniscus 50, to further shape it into the correct anatomic shape during final coagulation and solidification.

The present invention also provides a method of partial mensical repair 100 using a biologic partial meniscus 50 comprising inter alia the steps of: (i) providing a mold 20 resembling an excised/removed area 11 of a torn meniscus 10; (ii) providing a moldable allograft paste 60 within the mold 20 to form a biologic partial meniscus 50; and (iii) providing the biologic partial meniscus 50 at the repair site 90 to repair the meniscus and recreate meniscus 100. Preferably, the moldable allograft paste comprises meniscal cartilage in the form of morsellized, freeze-dried and/or desiccated meniscal cartilage (such as, for example, BioCartilage™)

In an exemplary and illustrative embodiment only, a method of arthroscopically-assisted surgical technique for partial mensical repair 100 using a construct 50 of the present invention comprises the steps of: (i) preparing a partial meniscal site by removing partial meniscal defect(s) 11 from a meniscus 10 to be repaired; (ii) providing a biologic partial meniscus graft 50 which is formed by mixing morcellized or dessicated meniscal cartilage with ACP, CACL or an autograft biologic equivalent, to create a moldable allograft paste 60; (iii) forming a biological resurfacing mold 20 (for example, a clear or a metal cap that covers at least the meniscal defect) that replicates/recreates the anatomical shape of the meniscus 10 to be replaced; (iv) injecting the allograft paste 60 into the mold 20, 20a and allowing the paste 60 to solidify to form the biologic partial meniscus graft 50; and (v) fixating the biologic partial meniscus graft 50 to the remaining/resected meniscus to recreate meniscus 100.

The biologic partial meniscus of the present invention may optionally comprise BMA, ACP, growth factors, additional antiseptic chemicals and/or antibiotics and/or electrolytes, or hormones or site-specific hybrid proteins (that promote or enhance the wound healing effectiveness of the growth factors), fibrin glue and adhesives, among others.

Growth factors may comprise proteinaceous factors, for example, which play a role in the induction or conduction of growth of tissue, ligaments, bone, cartilage or other tissues associated with bone or joints. In particular, the following growth factors contained in platelets are set forth below (and their effects):

PDGF (Platelet-derived growth factor)—Stimulates collagen synthesis, the formation of blood vessels and fibroblast proliferation; activation of macrophages and neutrophiles; activates TGF-β; attracts stem cells.

FGF (Fibroblast growth factor)—Stimulates the formation of blood vessels, collagen synthesis, wound contraction, matrix synthesis, epithelialisation.

TGF-β (Transforming growth factor β)—Reduces scar formation; reduces wound healing disturbances caused by corticoids; attracts fibroblasts and promotes their proliferation; stimulates collagen synthesis; promotes the secretion of FGF and PDGF by monocytes.

TGF-α (Transforming growth factor-α)—Stimulates mesenchymal, epithelial and endothelial cells.

EGF—(Epithelial Growth Factor)—Stimulates re-epithelialisation, the formation of new blood vessels and collagenase activity.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting.

What is claimed is:

1. A method of arthroscopic surgery, comprising:
conducting partial meniscectomy to remove at least part of a torn meniscus and to obtain a resected meniscus; and
replacing the at least part of the torn meniscus with a biological construct consisting essentially of allograft meniscal cartilage.

2. The method of claim 1, wherein the allograft meniscal cartilage consists of cartilage pieces.

3. The method of claim 1, wherein the allograft meniscal cartilage comprises hyaline cartilage pieces and at least one of ACP, PRP and fibrin glue.

4. The method of claim 1, wherein the biological construct is formed by the steps of:
mixing allograft meniscal cartilage with at least autologous conditioned plasma to obtain a moldable allograft paste;
providing the moldable allograft paste into a mold that replicates the shape and configuration of the torn meniscus to be repaired;
allowing the moldable allograft paste to solidify and form the biological construct; and
securing the biological construct to the resected meniscus to replicate the torn meniscus.

5. The method of claim 4, wherein the mold has a contour and configuration similar to those of the torn meniscus.

6. The method of claim 4, further comprising the step of providing at least one of a flexible strand or a mesh into the mold prior to the step of providing the moldable allograft paste into the mold, to create a biologic reinforcement and provide biomechanical strength to the biological construct.

7. The method of claim 6, wherein the mesh is a collagen mesh.

8. The method of claim 4, wherein the step of securing the biological construct to the resected meniscus further comprises providing at least one meniscal cinch.

9. The method of claim 4, wherein the step of securing the biological construct to the resected meniscus further comprises conducting at least one suturing step to securely attach the biological construct to the resected meniscus.

10. The method of claim 1, wherein the biological construct further comprises a biological component selected from the group consisting of blood, blood components, platelet-rich plasma, bone marrow aspirate and growth factors.

11. The method of claim 1, further comprising the step of inserting the biological construct to a repair site of the torn meniscus arthroscopically, by proving at least one cannulated, vented stent at the repair site and inserting the biological construct over the cannulated, vented stent.

12. A method of arthroscopically rebuilding a resected partial meniscus, comprising:
providing a partially resected meniscus obtained from a damaged meniscus at a repair site;
preparing a biological partial meniscal allograft consisting essentially of meniscal cartilage, the biological partial meniscal allograft being prepared by: providing cartilage pieces and mixing the cartilage pieces with at least autologous conditioned plasma to obtain an allograft moldable paste; inserting the allograft moldable paste into a reconstruction mold, the reconstruction mold having a contour and a configuration similar to the ones of the damaged meniscus; and allowing the autologous conditioned plasma to coagulate and the moldable paste to set and form the biological partial meniscal allograft;
removing the biological partial meniscal allograft from the reconstruction mold;
providing the biological partial meniscal allograft arthroscopically at the repair site; and
securing the biological partial meniscal allograft to the partially resected meniscus to replicate the anatomy of the damaged meniscus.

13. The method of claim 12, wherein the reconstruction mold is a clear mold or a metal mold.

14. The method of claim 12, further comprising the step of adding, to the allograft moldable paste, a component selected from the group consisting of growth factors, antiseptics, antibiotics and electrolytes.

15. The method of claim 12, wherein the damaged meniscus is a lateral or medial knee meniscus.

16. A graft for treatment of a damaged meniscus, the graft comprising a biological meniscal allograft consisting essentially of morselized, freeze-dried or desiccated meniscal cartilage.

17. The graft of claim 16, wherein the biological meniscal allograft further includes a biological component selected from the group consisting of platelet-rich plasma, autologous conditioned plasma and bone marrow aspirate.

18. The graft of claim 16, wherein the biological meniscal allograft further includes fibrin glue.

19. The graft of claim 16, wherein the biological meniscal allograft further includes growth factors, antiseptics, antibiotics and eletrolytes.

20. The graft of claim 16, wherein the damaged meniscus is part of a knee, a shoulder or a hip.
